(19) United States
(12) Patent Application Publication
(10) Pub. No.: US 2025/0296965 A1
(43) Pub. Date: Sep. 25, 2025

(54) USE OF RECOMBINANT TRANSFORMING GROWTH FACTOR BETA INDUCED (TGFBI) PROTEIN IN LATTICE CORNEAL DYSTROPHY (LCD)

(71) Applicant: EYE INSTITUTE OF SHANDONG FIRST MEDICAL UNIVERSITY, Qingdao (CN)

(72) Inventors: Lixin XIE, Qingdao (CN); Qingjun ZHOU, Qingdao (CN); Bining ZHANG, Qingdao (CN); Lin CONG, Qingdao (CN)

(73) Assignee: EYE INSTITUTE OF SHANDONG FIRST MEDICAL UNIVERSITY, Qingdao (CN)

(21) Appl. No.: 18/735,594

(22) Filed: Jun. 6, 2024

(30) Foreign Application Priority Data
Mar. 19, 2024 (CN) 202410311584.8

(51) Int. Cl. C07K 14/47; A61K 38/00; A61K 48/00; A61P 27/04; C12N 15/86

(52) U.S. Cl. CPC C07K 14/4703; A61K 48/00; A61P 27/04; C12N 15/86; A61K 38/00; C12N 2750/14143

(57) ABSTRACT

Use of a recombinant transforming growth factor beta induced (TGFBI) protein in lattice corneal dystrophy (LCD) is provided, belonging to the technical field of biomedicine. Use of a recombinant TGFBI protein and a recombinant TGFBI protein-expressing reagent in preparation of a medicament for treating LCD are provided. It is confirmed that dropping eye drops of the recombinant TGFBI protein can promote the repair of corneal epithelial damages in an LCD mouse model; corneal intrastromal injection of the recombinant TGFBI protein can promote the repair of corneal epithelial damages in the LCD mouse model; and limbal intrastromal injection of an adeno-associated virus (AAV) vector carrying a Tgfbi gene can promote the repair of epithelial damages in a TGFBI point mutation-carrying mouse. Therefore, the recombinant TGFBI protein-related reagents exhibit a potential to treat the LCD.

### CROSS-REFERENCE TO RELATED APPLICATIONS

This application claims foreign priority benefits under 35 U.S.C. § 119 (a)-(d) to Chinese patent application No. 202410311584.8 filed on Mar. 19, 2024, the disclosure of which is hereby incorporated herein by reference in its entirety.

### TECHNICAL FIELD

The present disclosure belongs to the technical field of biomedicine, and specifically relates to use of a recombinant transforming growth factor beta induced (TGFBI) protein in lattice corneal dystrophy (LCD).

### BACKGROUND

Lattice corneal dystrophy (LCD), an autosomal dominant disease caused by mutations in the pathogenic gene transforming growth factor beta induced (TGFBI), manifests as photophobia and pain, accompanied by decreased vision, lattice opacity in the corneal stroma, and recurrent erosion of the corneal epithelium. As the disease progresses, when symptomatic treatment with drugs fails to relieve symptoms, phototherapeutic keratectomy (PTK) is required or lamellar or penetrating keratoplasty is conducted after corneal stromal opacity worsens, and all of which are associated with persistent non-healing of the corneal epithelium after surgery. Therefore, targeting the corneal epithelial dysfunction caused by TGFBI gene mutation in LCD patients is a key to improving the prognosis of treatment. However, there is currently no precise treatment plan for the cause of LCD.

### SUMMARY

A purpose of the present disclosure is to provide use of a recombinant TGFBI protein in LCD. Etiological treatment based on the recombinant TGFBI protein to improve the function of limbal stem cells in LCD patients may fundamentally avoid the recurrence of LCD patients after receiving traditional treatment options.

The present disclosure provides use of a recombinant TGFBI protein in preparation of a medicament for treating LCD.

The present disclosure further provides use of a recombinant TGFBI protein-containing reagent in preparation of a medicament for treating LCD.

In some embodiments, the recombinant TGFBI protein-containing reagent includes an aqueous solution of the recombinant TGFBI protein or a gel prepared from the recombinant TGFBI protein.

In some embodiments, the use further includes modifying the recombinant TGFBI protein.

The present disclosure further provides use of a recombinant TGFBI protein-expressing reagent in preparation of a medicament for treating LCD.

In some embodiments, the recombinant TGFBI protein-expressing reagent includes a vector, an mRNA, or a plasmid for expressing a recombinant TGFBI protein.

In some embodiments, the use further includes modifying the vector, the mRNA, or the plasmid for expressing the recombinant TGFBI protein.

The present disclosure further provides a drug for treating LCD, including a recombinant TGFBI protein or a reagent expressing the recombinant TGFBI protein that serves as an active ingredient and a pharmaceutically acceptable auxiliary material.

In some embodiments, a dosage form of the drug includes an externally applied drug or an injection drug.

In some embodiments, a dosage form of the externally applied drug includes a microneedle, a liquid form, a cream, or a gel.

Beneficial effects: use of a recombinant TGFBI protein and a reagent expressing the recombinant TGFBI protein in preparation of a medicament for treating LCD are provided. It is confirmed in the examples that dropping eye drops of the recombinant TGFBI protein can promote the repair of corneal epithelial damages in a TGFBI point mutation-carrying mouse; corneal intrastromal injection of the recombinant TGFBI protein can promote the repair of corneal epithelial damages in the TGFBI point mutation-carrying mouse; and limbal intrastromal injection of an adeno-associated virus (AAV) vector carrying a Tgfbi gene can promote the repair of epithelial damages in the TGFBI point mutation-carrying mouse. That is to say, the present disclosure confirms an effectiveness of the recombinant TGFBI protein in eye drops in the treatment of epithelial damage repair in LCD, which may fundamentally reduce the risk of recurrence by conventional treatment of LCD. Therefore, the recombinant TGFBI protein-related reagents exhibit a potential to treat the LCD.

### BRIEF DESCRIPTION OF THE DRAWINGS

FIG. 1 shows a schematic structural diagram of the AAV vector for overexpressing the recombinant TGFBI protein;

FIG. 2 shows the results of dropping eye drops of the TGFBI recombinant protein for promoting the repair of corneal epithelial damages in TGFBI point mutation-carrying mice;

FIG. 3 shows the results of corneal intrastromal injection of the TGFBI recombinant protein for promoting the repair of corneal epithelial damages in TGFBI point mutation-carrying mice;

FIG. 4 shows that 3 d after AAV corneal limbus injection at 4 spots, a uniform and stable GFP expression was seen in corneal epithelial cells; and

FIG. 5 shows the results of corneal damage repair by corneal intrastromal injection of AAV-Tgfbi and AAV-Con from 24 h to 72 h after corneal epithelial scraping, and the epithelial repair of the AAV-Tgfbi injection group was significantly faster than that of the AAV-Con injection group.

### DETAILED DESCRIPTION

As required, detailed embodiments of the present disclosure are disclosed herein; however, it is to be understood that the disclosed embodiments are merely exemplary of the disclosure that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present disclosure.

The present disclosure provides use of a recombinant TGFBI protein in preparation of a medicament for treating LCD.

In the present disclosure, the recombinant TGFBI protein has an amino acid sequence preferably as set forth in NP_000349.1 in NCBI protein database; in the examples, a commercially available product, SinoBiological, Cat: 10569-H08H, 100 μg, is preferably used as an example for explanation.

In the present disclosure, the recombinant TGFBI protein is a water-soluble protein and can be dissolved in water; in the examples, ultrapure water is preferably used to dissolve the recombinant TGFBI protein. It is confirmed in the examples that dropping a solution of the TGFBI recombinant protein can promote the repair of corneal epithelial damages in TGFBI point mutation-carrying mice, and therefore it can be used to prepare a medicament for treating LCD.

The present disclosure further provides use of a recombinant TGFBI protein-containing reagent in preparation of a medicament for treating LCD.

In the present disclosure, the recombinant TGFBI protein-containing reagent includes preferably an aqueous solution of the recombinant TGFBI protein or a gel prepared from the recombinant TGFBI protein. The recombinant TGFBI protein can also be modified or the dosage form of the aqueous solution can be changed into a hydrogel, so as to exert the effects of sustained release and wound protection.

The present disclosure further provides use of a recombinant TGFBI protein-expressing reagent in preparation of a medicament for treating LCD.

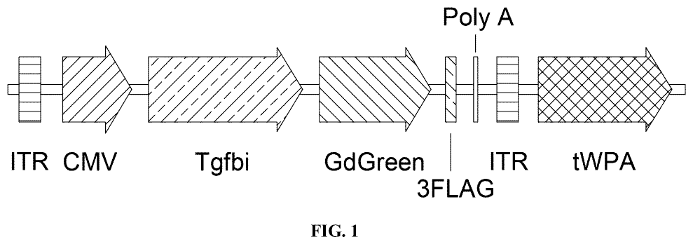

In the present disclosure, the reagent for expressing the recombinant TGFBI protein includes preferably a vector, an mRNA, and a plasmid for expressing the recombinant TGFBI protein. The vector, mRNA, or plasmid for expressing the recombinant TGFBI protein can also be modified, such as by modifying the capsid or modifying the plasmid, thereby improving the transfection and expression efficiency of limbal stem cells. The vector is preferably an AAV vector; in the examples, the AAV vector (FIG. 1) for overexpressing the recombinant TGFBI protein is preferably used as an example for explanation. Moreover, the AAV vector overexpressing the recombinant TGFBI protein is purchased from OBiO Co., Ltd. AAV-Con, GL3050, 1.54×10¹³ μg/mL; AAV-Tgfbi, H29917, 5.22×10¹² μg/mL.

The present disclosure further provides a drug for treating LCD, including a recombinant TGFBI protein or a reagent for expressing the recombinant TGFBI protein that serves as an active ingredient and a pharmaceutically acceptable auxiliary material.

In the present disclosure, the recombinant TGFBI protein and/or the reagent for overexpressing the recombinant TGFBI protein exerts a therapeutic effect through eye drops dropping, subconjunctival injection, or limbal intrastromal injection. Therefore, the medicament can be prepared as an externally applied drug or an injection drug, including eye drops, sustained-release drugs, hydrogels, microneedles, or other dosage forms for increasing corneal adhesion containing modified recombinant TGFBI protein, and the likes as well as into a plasmid, mRNA, and AAV vector carrying an exogenous recombinant Tgfbi gene that is capable of being transfected into the ocular surface tissues (cornea, conjunctiva, meibomian glands, lacrimal glands, and the likes) and expressing the recombinant TGFBI protein by regulating the upstream or downstream products involved in a TGFBI protein signaling pathway. The dosage form of the externally applied drug preferably includes a microneedle, a liquid form, a cream, or a gel.

To further illustrate the present disclosure, the use of a recombinant TGFBI protein in preparation of a medicament for treating LCD provided by the present disclosure is described in detail below in connection with examples, but these examples should not be construed as limiting the claimed scope of the present disclosure.

### Example 1

1. TGFBI point mutation-carrying mice (hereinafter also referred to as “TGFBI mice”)

An R124 site of the mouse Tgfbi gene exon4 was mutated to C124 using a Cas9 gene editing method, that is, R124C. The obtained point mutation-carrying mice could develop symptoms such as corneal opacity and delayed repair of corneal epithelial damages during development, and were highly consistent with clinical patients suffered from LCD. The Tgfbi-R124C point mutation-carrying mice were constructed by Beijing ViewSolid Biotech Co., Ltd.

2. Preparation of recombinant TGFBI protein eye drops (0.25 μg/μL)

100 μg of a lyophilized powder of the recombinant TGFBI protein was dissolved in 400 μL of sterile water for injection according to the instructions, and diluted to 0.25 μg/μL, that is, 250 μg/mL.

3. 2.5 mm of corneal epithelium in the central area of the TGFBI point mutation-carrying mice was scraped; and the mice were dropped with eye drops of PBS in the control group, and of recombinant TGFBI protein (10 μL, 0.25 μg/μL) in the experimental group, twice daily; the repair status of corneal epithelial damages was regularly observed by fluorescein sodium staining.

The mice were anesthetized with 0.3 mL of 0.6% sodium phenobarbital through intraperitoneal injection, anesthetized mice were placed on an operating table, and both eyes were locally anesthetized using proparacaine hydrochloride eye drops. The mouse orbit was gently pressed with microscopic flat forceps to expose the eyeball, a 2.5 mm diameter trephine was used to gently press the center of the cornea to make a 2.5 mm diameter indentation on the corneal epithelium, the corneal epithelium in the central area was scraped along the indentation with an Algerbush II electric epithelial scraper, and tobramycin and dexamethasone ophthalmic ointment was applied thereon. The mice were dropped with eye drops of PBS in the control group, and of recombinant TGFBI protein eye drops (10 μL, 0.25 μg/μL) in the experimental group, twice daily; the repair status of corneal epithelial damages was regularly observed by fluorescein sodium staining.

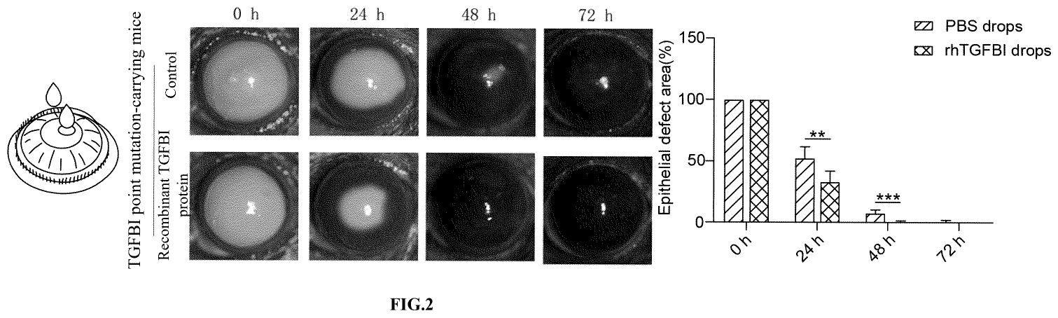

The results were shown in FIG. 2. The epithelial repair of the mice in the group using the recombinant TGFBI protein eye drops was significantly faster than that in the control group, and the differences were statistically significant.

24 h after scraping the corneal epithelium, it was seen that the epithelial defect area in the recombinant TGFBI protein eye drops-dropping group was significantly smaller than that in the PBS control group, and the corneal damage repair was accelerated. 48 h after scraping the corneal epithelium, the epithelium of the recombinant TGFBI protein eye drops-dropping group was basically repaired completely, while the epithelium of the PBS control group still remained some defects. The differences between the experimental group and the control group were statistically significant.

4. TGFBI mice were subjected to corneal intrastromal injection 1 d before corneal epithelial scraping, where the control group was injected with PBS (2 μL), while the experimental group was injected with recombinant TGFBI protein (2 μL, 0.25 μg/μL). After 1 day, 2.5 mm of corneal epithelium in the central area was scraped, and the repair status of corneal epithelial damages was regularly observed by fluorescein sodium staining.

1 d before scraping the corneal epithelium in the central area, TGFBI mice were subjected to intrastromal injection with recombinant TGFBI protein and PBS, respectively. As described above, the mice were anesthetized with 0.6% phenobarbital sodium, anesthetized mice were placed on the operating table, and an 11-0 suture needle was used to make a lamellar puncture of about 0.5 mm long in the central area of the cornea toward the center of the cornea, where a Hamilton 36G microinjection needle was used to aspirate recombinant TGFBI protein (0.25 μg/μL, 2 μL) in the experimental group, while a Hamilton 36G microinjection needle was used to aspirate PBS (2 μL) in the control group. The recombinant TGFBI protein was gently injected into the corneal stroma by reaching along the puncture opening while keeping the needle beveled down, and tobramycin and dexamethasone ophthalmic ointment was applied thereon. On the 2nd day, 2.5 mm diameter epithelium in the central area of the cornea was scraped off based on the method above. The repair status of corneal epithelial damages was regularly observed by fluorescein sodium staining.

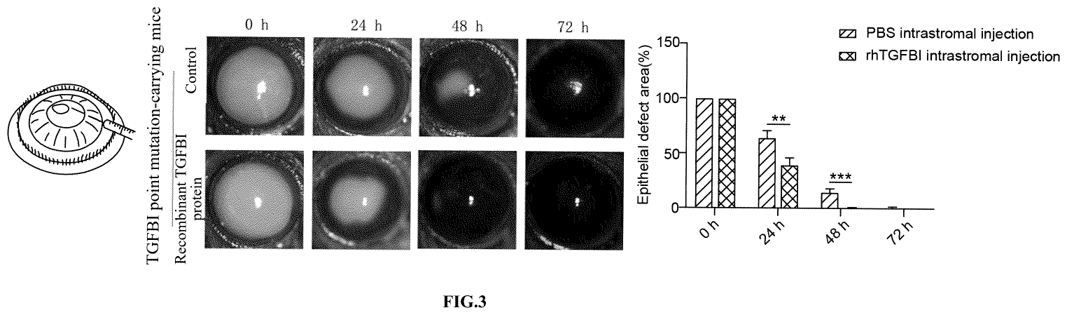

The results were shown in FIG. 3. The epithelial repair of mice in the recombinant TGFBI protein intrastromal injection group was significantly faster than that in the control group, and the differences were statistically significant.

24 h after scraping the corneal epithelium, it was seen that the epithelial defect area in the recombinant TGFBI protein intrastromal injection group was significantly smaller than that in the PBS intrastromal injection group, and the corneal damage repair was accelerated. 48 h after scraping the corneal epithelium, the epithelium of the recombinant TGFBI protein injection group was basically repaired completely, while the epithelium of the PBS injection group still remained some defects. The differences between the experimental group and the control group were statistically significant.

5. 3 d before epithelial scraping, TGFBI mice were injected in the peripheral corneal stroma at 4 spots, where the control group was injected with AAV-Con (OBiO, GL3050, 1×10⁹ μg/μL, 4 μL), while the experimental group was injected with AAV-Tgfbi (OBiO, H29917, 1×10⁹ μg/μL, 4 μL). After 3 d, 2.5 mm of corneal epithelium in the central area was scraped, and the repair status of corneal epithelial damages was regularly observed by fluorescein sodium staining.

3 d before scraping the corneal epithelium in the central area, TGFBI mice were injected with AAV-Tgfbi in the experimental group and AAV-Con in the control group at 4 spots in the corneal limbus to transfect limbal stem cells and corneal epithelial cells. As described above, the mice were anesthetized with 0.6% sodium phenobarbital, anesthetized mice were placed on the operating table, and 11-0 suture needles were used to make a 0.5 mm long lamellar puncture horizontally to the corneal limbus at the upper, lower, nasal, and temporal of the corneal limbus, respectively; where a Hamilton 36G microinjection needle was used in the experimental group to aspirate AAV-Tgfbi (OBiO, H29917, 1×10⁹ μg/μL, 4 μL), while a Hamilton 36G microinjection needle was used in the control group to aspirate AAV-Con (OBIO, GL3050, 1×10⁹ μg/μL, 4 μL), the needle was inserted into each lamellar puncture opening and inject 1 μL of virus solution, and tobramycin and dexamethasone ophthalmic ointment was applied thereon. 3 d after the AAV intrastromal injection, 2.5 mm diameter epithelium in the central area of the cornea was scraped off based on the method above. The repair status of corneal epithelial damages was regularly observed by fluorescein sodium staining.

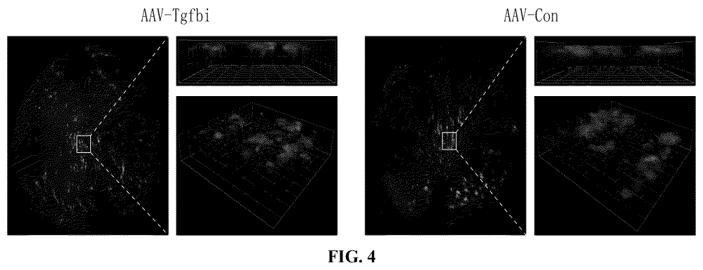

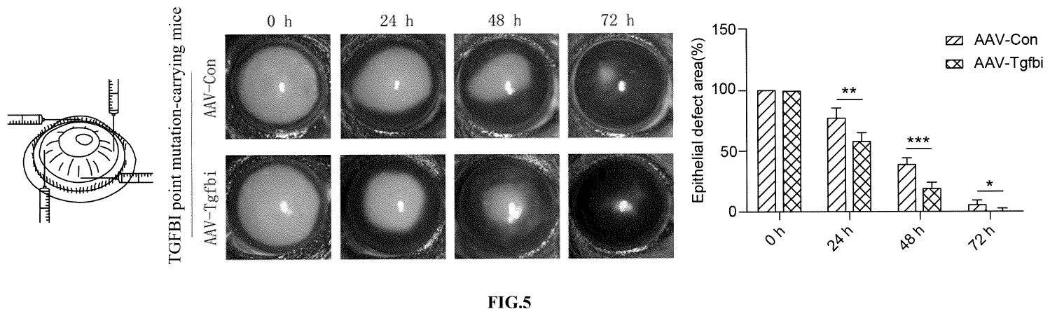

The results were shown in FIG. 4 and FIG. 5. The epithelial repair of the AAV-Tgfbi injection group was significantly faster than that of the AAV-Con injection group, and the differences were statistically significant.

FIG. 4 showed that 3 d after AAV corneal limbus injection at 4 spots, relatively uniform fluorescent protein expression was seen in the cornea, and an enlarged 3D picture of the central area showed that fluorescent protein expression was concentrated in the corneal epithelium. This suggested that the corneal epithelium began to express the exogenous target gene carried by AAV 3 d after AAV transfection, so as to exert a therapeutic effect. FIG. 5 showed that 24 h and 48 h after the corneal epithelial was scraped, the epithelial defect area in the AAV-Tgfbi intrastromal injection group was significantly smaller than that in the AAV-Con group, suggesting that the AAV-Tgfbi group had accelerated corneal damage repair. 72 h after the corneal epithelium was scraped, the epithelium in the AAV-Tgfbi group had been basically repaired completely, while the epithelium in the AAV-Con group still remained some defects. The difference between the two groups was statistically significant.

Although the above examples have described the present disclosure in detail, it is only a part of, not all of, the examples of the present disclosure. Other examples may also be obtained by persons based on the present examples without creative efforts, and all of these examples shall fall within the protection scope of the present disclosure.

What is claimed is:

1. A method for treating a subject with lattice corneal dystrophy (LCD), wherein a recombinant transforming growth factor beta induced (TGFBI) protein, a recombinant TGFBI protein-containing reagent or a recombinant TGFBI protein-expressing reagent is administrated to the subject in need thereof.

2. The method according to claim 1, wherein the recombinant TGFBI protein-containing reagent comprises an aqueous solution of the recombinant TGFBI protein or a gel prepared from the recombinant TGFBI protein.

3. The method according to claim 1, further comprising modifying the recombinant TGFBI protein.

4. The method according to claim 1, wherein the recombinant TGFBI protein-expressing reagent is a vector, an mRNA, or a plasmid for expressing a recombinant TGFBI protein.

5. The method according to claim 4, further comprising modifying the vector, the mRNA, or the plasmid for expressing the recombinant TGFBI protein.

6. A drug for treating LCD, comprising a recombinant TGFBI protein or a recombinant TGFBI protein-expressing reagent that serves as an active ingredient and a pharmaceutically acceptable auxiliary material.

7. The drug according to claim 6, wherein a dosage form of the drug is an externally applied drug or an injection drug.

8. The drug according to claim 7, wherein the dosage form of the externally applied drug is a microneedle, a liquid form, a cream, or a gel.
